(19) United States
(12) Patent Application Publication
(10) Pub. No.: US 2004/0191169 A1
(43) Pub. Date: Sep. 30, 2004

(54) Methods of protection from toxicity of alpha emitting elements during radioimmunotherapy

(76) Inventors: David Scheinberg, New York, NY (US); Michael R. McDevitt, Bronx, NY (US); Jaspreet Jaggi, New York, NY (US)

Correspondence Address: Benjamin Aaron Adler, ADLER & ASSOCIATES, 8011 Candle Lane, Houston, TX, 77071, US

(21) Appl. No.: 10/806,905

(22) Filed: Mar. 23, 2004

Related U.S. Application Data
(60) Provisional application No. 60/457,503, filed on Mar. 25, 2003.

(51) Int. Cl. A61K 51/00

(52) U.S. Cl. 424/1.49

(57) ABSTRACT

Provided herein are methods of reducing nephrotoxicityfrom at least one alpha particle-emitting daughter of actinium-225 during radioimmunotherapeutic treatment for a pathophysiological condition, methods of improving radioimmunotherapeutic treatment of cancer and methods of increasing the therapeuticindex of an actinium-225 radioimmunoconjugate during treatment of a pathophysiological condition. Adjuvants effective for preventing accumulation of ²²⁵Ac daughters within the kidneys are administered during treatment with an actinium-225 radioimmunoconjugate to reduce nephrotoxicity. Examples of adjuvants are chelators, diuretics and/or competitive metal blockers.

### CROSS-REFERENCE TO RELATED APPLICATIONS

This nonprovisional application claims benefit of priority of provisional application U.S. Serial No. 60/457,503, filed Mar. 25, 2003, now abandoned.

### FEDERAL FUNDING LEGEND

This invention was produced in part using funds obtained through grant R01-CA 55349 from the National Institutes of Health. Consequently, the federal government has certain rights in this invention.

### BACKGROUND OF THE INVENTION

1. Field of the Invention

The present invention relates generally to the fields of radioimmunotherapy and cancer treatment. Specifically, the present invention provides methods of protecting an individual from toxicity of alpha particle-emitting elements during radioimmunotherapy.

2. Description of the Related Art

Monoclonal antibody (mAb) based therapies are ideally applicable to the hematopoietic neoplasms (1) because of readily accessible neoplastic cells in the blood, marrow, spleen and lymph nodes which allow rapid and efficient targeting of specific mAb's. The well characterized immunophenotypes of the various lineages and stages of hematopoietic differentiation has enabled identification of antigen targets for selective binding of mAb to neoplastic cells while relatively sparing other necessary hematopoietic lineages and progenitor cells. Similar work is now being carried out for a variety of solid cancers as well.

In some models of leukemia, specific uptake of antibodies onto target cells can be demonstrated within minutes, followed by losses of mAb from the cells by modulation (2,3). Similar modulation has been seen in pilot studies in acute leukemia in humans (4-7). Based on this biology and pharmacokinetics, it has been proposed that mAb tagged with short-lived nuclides emitting short-ranged, high linear energy transfer (LET) alpha particles (8-9) or short-ranged auger electrons (10-11), may be effective in therapy. These short-ranged particles may be capable of single cell kill while sparing bystanders.

Pilot trials conducted in patients with hematopoieticcancers (4-7,12) have demonstrated the ability of mAb to bind to target cells and have also highlighted the problems of antigen modulation, antigen heterogeneity, tumor burden and human anti-mouse antibody (HAMA) response (4-7,12-16). Some short-lived major tumor responses were seen in these early trials with non-cytotoxic antibodies. More consistent responses were next achieved in recent trials using cytotoxic mAb and isotope tagged mAb (17-24). Two antibodies to CD20 are now approved for the treatment of non-Hodgkin's lymphoma (24-26). Recently, one antibody for treating AML and one for CLL were also approved. (26-28). A large systematic in vivo study of various antibody-based immuno-therapies in acute myelogenous leukemia with more than 300 treated patients has been conducted (4,19,21,29-31).

The expression of the CD33 antigen is restricted to myelogenous leukemias and myeloid progenitor cells, but not to other normal tissues or ultimate bone marrow stem cells (32-35). In summary it has been demonstrated that HuM195 is highly specific for myeloid leukemia cells both in vitro and in vivo; HuM195 does not react with tissue or cells of other types or neoplastic cells not of myeloid origin. HuM195 reacts with early myeloid progenitors, but not stem cells, and reacts with monocytes and dendritic cells, but no other mature hematopoietic elements. HuM195 mAbs have high affinities, i.e., on the order of 10⁻⁹ to 10⁻¹⁰ M. M195 mAbs are internalized into target cells after binding.

A series of early studies defined the pharmacology, safety profile, biodistribution, immunobiology, and activity of various M195 agents. M195 showed targeting to leukemia cells in humans (4). Adsorption of M195 onto leukemic target cells in vivo was demonstrated by biopsy, pharmacology, flow cytometry, and imaging; saturation of available sites occurred at doses 5 mg/m². The entire bone marrow was specifically and clearly imaged beginning within minutes after injection; optimal imaging occurred at 5-10 mg dose levels. Bone marrow biopsies demonstrated significant dose-related uptake of M195 as early as 1 hour after infusion in all patients with the majority of the dose found in the marrow. M195 was rapidly modulated with a majority of the bound IgG being internalized into target cells in vivo.

Other trials showed that radiolabeled beta emitting M195, with either I-131 or Y-90, can effect up to 100% cytoreduction of leukemic cells (19). Most patients had reduction in their leukemia burden with prolonged marrow hypoplasia achieved at higher dose levels. These patients were taken to BMT and nearly all achieved CR with several ultimately cured.

A wide variety of nuclides suitable for mAb-guided radiotherapy have been proposed (12). Depending on the particular application, three classes of radionuclides may prove therapeutically useful in leukemia (9-11, 17, 19-23,36-44): β-emitters (¹³¹I, ⁹⁰Y) with long range (1-10 mm) emissions are probably limited to settings of larger tumor burden where BMT rescue is feasible. Alpha-emitters (²¹³Bi, ²¹¹At) with very high energy but short ranges (0.05 mm) may allow more selective ablation (37-51). Auger emitters (¹²³I, ¹²⁵I) which act only at subcellular ranges (<1 micron) will yield single cell killing but only if internalized.

Radioimmunotherapy has advanced tremendously in the last 20 years with the development of more sophisticated carriers, as well as of radionuclides optimized for a particular cancer and therapeutic application (52). Radioimmunotherapy (RIT) with alpha particle emitting radionuclides is advantageous because alpha particles have high LET and short path lengths (50-80 μm) (53-57). Therefore, a large amount of energy is deposited over a short distance, which renders alpha particles extremely cytotoxic with a high relative biological effectiveness (55-56). Little collateral damage to surrounding normal, antigen-negative cells occurs (57-59). A single traversal of densely ionizing, high energy alpha particle radiation through the nucleus, may be sufficient to kill a target cell (60). In addition, the double stranded DNA damage caused by alpha particles is not easily repaired by the cells, and this cytotoxicity is largely unaffected by the oxygen status and cell-cycle position of the cell (53).

The results of pre-clinical studies with alpha particle emitting ²²⁵Ac atomic nanogenerators have generated optimism for their human clinical use (61-62). ²²⁵Ac has a sufficiently long half-life (10 days) for feasible use and it decays to stable Bismuth-209 via six atoms, yielding a net of four alpha particles (FIG. 1). This permits delivery of radiation even to the less readily accessible cells and also for the radiopharmaceutical to be shipped world-wide (61).

²²⁵Ac is successfully coupled to internalizing monoclonal antibodies using DOTA (1,4,7,10-tetraazacyclododecane-1,4,7,10-tetraacetic acid) as the chelating moiety. The ²²⁵Ac-DOTA-antibody construct acts as a tumor-selective, molecular-sized, in-vivo atomic generator, i.e., a targetable nanogenerator, of alpha particle emitting elements (61). The ²²⁵Ac-DOTA-antibody constructs are stable in-vivo and have been shown to be safe and potent anti-tumor agents in mouse models of solid prostatatic carcinoma, disseminated lymphoma and intraperitoneal ovarian cancer (61-62). The safety of ²²⁵Ac-HuM195 and ²²⁵Ac-3F8 at low doses, has been demonstrated in primates (63).

²²⁵Ac decays via its alpha-emitting daughters, Francium-221 (²²¹Fr), Astatine-217 (217 At) and Bismuth-213 (²²¹Bi) to stable, non-radioactive ²⁰⁹Bi (58,60,63). These daughters, once formed, are unlikely to associate with the antibody-DOTA construct due to high atomic recoil-energy as a result of alpha decay (65), possible rupture of the chelate and different chemical properties of the daughters. The daughters generated and retained inside the cancer cell after internalization of the ²²⁵Ac labeled antibody, add to its cytotoxiceffect (61). Although this property greatly enhances the potency of the ²²⁵Ac nanogenerators, it could also result in toxicity as the systemically released radioactive daughters may get transported to and irradiate the normal tissues. The ²²⁵Ac-immunoconjugate is stable in vivo and the daughters released inside the target cell remain internalized (61). However, the daughters released from the circulating ²²⁵Ac nanogenerator, tend to distribute independently of the parent construct (63).

Tumor burden is an important determinant in the biodistribution of the antibody (16, 65). However, the free daughters produced in the vasculature from the circulating unbound antibody or the antibody bound to the surface of a target cell, could diffuse or be transported to various target organs where they can accumulate and cause radiotoxicity. Bismuth is known to accumulate in the renal cortex (66-69). It has been observed that after injection in mice, francium rapidly accumulates in the kidneys (unpublished result). Francium distribution in the body has not been described due to its short half-life that makes experimental study difficult (69).

Monkeys injected with escalating doses of the untargeted ²²⁵Ac nanogenerator developed a delayed radiation nephropathy manifesting as anemia and renal failure (63). Therefore, a possible hindrance to the development of these agents as safe and effective cancer therapeutics is likely to be their nephrotoxicity. By preventing the renal accumulation of the radioactive daughters or by accelerating their clearance from the body, the therapeutic-index of the ²²⁵Ac nanogenerator could be enhanced.

Astatine-217 has the shortest half-life of 32 ms of the alpha-emitting daughters of ²²⁵Ac. It decays almost instantaneously to ²¹³Bi. ²¹³Bi and ²²¹Fr have relatively longer half-lives of 45.6 min. and 4.9 min., respectively, and therefore, have the potential to cause radiation damage (61,59). The presence of bismuth-binding, metallothionein-like proteins in the cytoplasm of renal proximal tubular cells, makes the kidney a prime target for the accumulation of free, radioactive bismuth (66-68). Dithiol chelators have been shown to chelate bismuth and enhance its excretion in various animal as well as human studies (64,69,71-72). Dithiol chelators also enhanced the total body clearance of the gamma emitting tracer, ²⁰⁶Bi acetate (12). Chelators such as ethylenediamine tetraacetic acid (EDTA) or diethylenetriamine pentaacetic acid (DTPA) also may chelate such metals. Ca-DTPA has been used in the U.S. as a chelating agent for plutonium and other transuranic elements (73-74).

²²¹Fr is another potentially toxic daughter of ²²⁵Ac. Francium, like sodium and potassium, is an alkali metal. Furosemide and thiazide diuretics are known to increase urine output and accelerate the elimination of sodium and potassium in urine, by inhibiting their reabsorption in different segments of the nephron (75).

The inventors have recognized a need in the art to improve the safe and efficacious use of ²²⁵Ac as a stable and extraordinarily potent tumor-selective molecular sized generator in both established solid carcinomas or in disseminated cancers. Specifically, the prior art is lacking in methods of using, individually or in combination, adjuvant chelation, diuresis or competitive metal blockade to reduce nephrotoxicity from ²²⁵Ac daughters generated during radioimmunotherapy. The present invention fulfills this long-standing need and desire in the art.

### SUMMARY OF THE INVENTION

The present invention is directed to a method of reducing nephrotoxicity in an individual during radioimmunotherapeutic treatment of a pathophysiological condition. A pharmacologically effective dose of at least one adjuvant effective for preventing accumulation of a metal in kidneys and an actinium-225 radioimmunoconjugate to treat the pathophysiological condition are administered to the individual. Accumulation of an alpha particle-emitting daughter of the actinium-225 within the kidneys of the individual is prevented via interaction between the adjuvant and the ²²⁵Ac daughter or the kidney tissue or a combination thereof thereby reducing nephrotoxicity during the radioimmunotherapeutic treatment.

The present invention is directed to related methods of reducing nephrotoxicity in an individual by administering a diuretic alone or in combination with the chelator and administering an actinium-225 radioimmunoconjugate to treat the pathophysiological condition. The chelator scavenges bismuth-213 daughters of actinium-225. The diuretic inhibits reabsorption of francium-211 daughters of actinium-225 within the kidneys to prevent accumulation thereof to reduce nephrotoxicity.

The present invention also is directed to a method of improving radioimmunotherapeutic treatment of cancer in an individual. As described above a pharmacologically effective dose of a chelator and an actinium-225 radioimmunoconjugate are administered individually. The chelator scavenges bismuth-213 daughters of the actinium-225 to reduce nephrotoxicity in the individual during treatment thereby increasing the therapeutic index of the actinium-225 to improve the treatment for cancer.

The present invention also is directed to related methods of improving radioimmunotherapeutic treatment of cancer by reducing nephrotoxicity in the individual during treatment thereby increasing the therapeutic index of the actinium-225 to improve the treatment for the cancer. A diuretic alone or in combination with the chelator and an actinium-225 radioimmunoconjugate are administered individually to the individual. The chelator functions as described above. The diuretic inhibits renal uptake of francium-211 daughters within the kidneys to reduce nephrotoxicity.

The present invention is directed further to a method of increasing the therapeutic index of an actinium-225 radioimmunoconjugate during treatment of a pathophysiological condition in an individual. Renal uptake of at least one alpha particle-emitting daughter of actinium-225 is inhibited whereby nephrotoxicity is reduced during the treatment thereby increasing the therapeutic index of said actinium-225 radioimmunoconjugate. In related methods inhibition of renal uptake of ²²⁵Ac daughters is accomplished by administering a pharmacologically effective amount of an adjuvant comprising a chelator to scavenge the ²²⁵Ac daughters therewith or of a diuretic to inhibit reabsorption of the ²²⁵Ac daughters within a kidney or of a competitive metal blocker to prevent binding of ²¹³Bi within a kidney or a combination of a chelator, a diuretic and the competitive metal blocker.

Other and further aspects, features, and advantages of the present invention will be apparent from the following description of the presently preferred embodiments of the invention. These embodiments are given for the purpose of disclosure.

### BRIEF DESCRIPTION OF THE DRAWINGS

The appended drawings have been included herein so that the above-recited features, advantages and objects of the invention will become clear and can be understood in detail. These drawings form a part of the specification. It is to be noted, however, that the appended drawings illustrate preferred embodiments of the invention and should not be considered to limit the scope of the invention.

FIG. 1 depicts a simplified Ac-225 generator to Bi-213 decay scheme, yielding 4 net alphas. The half-lives are shown in italics.

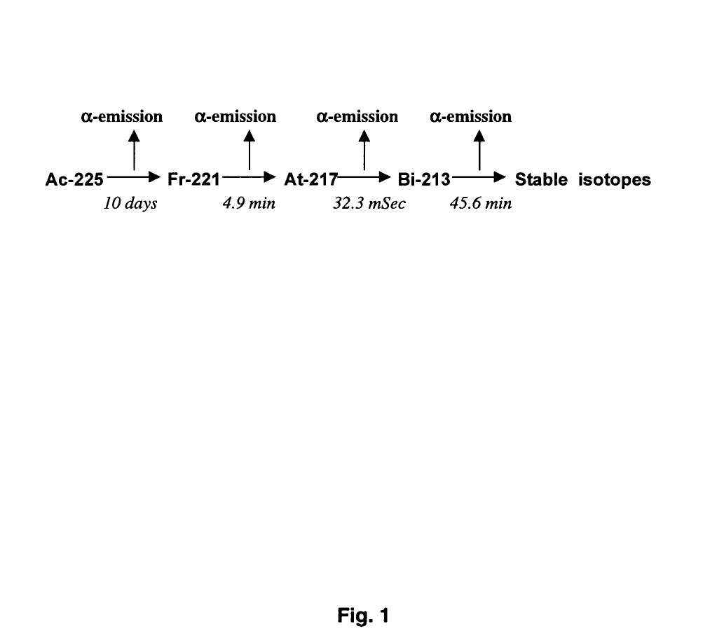

FIG. 2 depicts the structures of 2,3 dimercapto-1-propanesulfonic acid (DMPS) and meso 2,3 dimercaptosuccinic acid (DMSA)

FIGS. 3A-3B compare the effect of dithiol chelators on ²¹³Bi distribution in kidneys and blood. FIG. 3A compares reduction in the renal ²¹³Bi activity by DMPS or DMSA treatment at 6 hours and 72 hours post-injection. The renal ²²¹Fr activity is unchanged at both time-points. FIG. 3B compares the increase in the ²¹³Bi activity in blood by chelation therapy with DMPS or DMSA at 6 hours and 72 hours post injection. Data are mean (SE). % ID/g=percentage of injected dose per gram of tissue.

FIGS. 4A-4B depict the effect of diuresis or a combination of metal chelation and diuresis on renal ²²¹Fr and ²¹³Bi activity. FIG. 4A shows the reduction in the 24 hour renal ²²¹Fr and ²¹³Bi activities by furosemide and chlorothiazide (CTZ) treatment. FIG. 4B shows the reduced renal accumulation of ²²¹Frand ²¹³Bi at 24 hours post-injection by combination therapy with DMPS and furosemide or CTZ. Data are mean (SE). % ID/g=percentage of injected dose per gram of tissue.

FIG. 5 depicts the effect of competitive metal blockade on ²²⁵Ac daughter distribution and shows the reduction in the renal ²¹³Bi activity by bismuth subnitrate (BSN) at 6 hours and 24 hours post-injection.

FIGS. 6A-6C depict the effect of tumor burden on ²²⁵Ac daughter distribution. FIG. 6A compares the percentage of human-CD20 cells in the bone marrow of a “high burden” and a “low burden” animal to that of a non tumor-bearing mouse of the same strain. FIG. 6B shows the reduction in the ratio of kidney to femur activity for ²²⁵Ac and ²¹³Bi in animals with higher tumor burden. DMPS treatment further reduced the kidney to femur activity ratio for ²¹³Bi. FIG. 6C shows the reduction in the renal ²¹³Bi activity by the presence of higher tumor burden, and further enhancement of the effect by concomitant DMPS treatment. Error bars denote SE. % ID/g=percentage of injected dose per gram of tissue.

FIG. 7 depicts the biodistribution of [Ac]Hum195 at 24 hours in DMPS-treated and untreated monkeys.

### DETAILED DESCRIPTION OF THE INVENTION

In one embodiment of the present invention there is provided a method of reducing nephrotoxicity in an individual during radioimmunotherapeutic treatment of a pathophysiological condition comprising administering a pharmacologically effective dose of at least one adjuvant effective for preventing accumulation of a metal in kidneys; administering an actinium-225 radioimmunoconjugate to treat the pathophysiological condition; and preventing accumulation of alpha particle-emitting daughters of the actinium-225 within the kidneys of the individual via interaction between the adjuvant and the ²²⁵Ac daughters or the kidney tissue or a combination thereof thereby reducing nephrotoxicity during the radioimmunotherapeutic treatment. In an aspect of this embodiment the adjuvant(s) may be administered prior to administering the actinium-225 radioimmunoconjugate with the adjuvant(s) continuing to be administered after the actinium-225 radioimmunoconjugate.

In other aspects of this embodiment the adjuvant maybe a chelator, a diuretic, a competitive metal blocker or a combination of these. Representative examples of a chelator are 2,3 dimercapto-1-propane sulfonic acid, meso 2,3-dimercapto succinic acid, diethylenetriamine pentaacetic acid, calcium diethylenetriamine pentaacetic acid, or zinc diethylenetriamine pentaacetic acid. Examples of a diuretic are furosemide, chlorthiazide, hydrochlorothiazide, bumex or other loop diuretic. The competitive metal blocker may be bismuth subnitrate or bismuth subcitrate. In these aspects the ²²⁵Ac daughter may be bismuth-213, francium-221 or a combination thereof.

In all aspects the actinium-225 radioimmunoconjugate may comprise an actinium-225 bifunctional chelant and a monoclonal antibody. An example of such a radioimmunoconjugate is [²²⁵Ac] DOTA-HuM195. Further to all aspects the pathophysiological condition may be a cancer or an autoimmune disorder. The cancer may be a solid cancer, a disseminated cancer or a metastatic cancer. A representative cancer is myeloid leukemia.

In a related embodiment there is provided a method of reducing nephrotoxicity in an individual during radioimmunotherapeutic treatment of a pathophysiological condition comprising administering a pharmacologically effective dose of a chelator; administering an actinium-225 radioimmunoconjugate to treat the cancer; and preventing accumulation of bismuth-213 daughters of the actinium-225 within the kidneys of the individual by scavenging thereof with the chelator thereby reducing nephrotoxicity during the radioimmunotherapeutic treatment.

Further to this embodiment the method comprises administering a pharmacologically effective dose of a diuretic and preventing accumulation of francium-211 daughters of the actinium-225 within the kidneys of the individual by inhibiting reabsorption of francium-211 therein with the diuretic thereby reducing nephrotoxicity during the radioimmunotherapeutic treatment.

In another related embodiment there is provided a method of reducing nephrotoxicity in an individual during radioimmunotherapeutic treatment of a pathophysiological condition comprising administering a pharmacologically effective dose of a diuretic; administering an actinium-225 radioimmunoconjugate to treat the cancer; and preventing accumulation of francium-211 daughters of the actinium-225 within the kidneys of the individual by inhibiting reabsorption of francium-211 therein with the diuretic thereby reducing nephrotoxicity during the radioimmunotherapeutic treatment.

In all of these related embodiments the chelators and the diuretics are as described supra. Additionally, the points of administration of the chelator and/or the diuretic during treatment are as described supra. Furthermore, in these related embodiments the ²²⁵Ac radioimmunoconjugate and the cancers treated are as described supra.

In another embodiment of the present invention there is provided a method of improving radioimmunotherapeutic treatment of a cancer in an individual, comprising administering a pharmacologically effective dose of a chelator; administering an actinium-225 radioimmunoconjugate; and scavenging bismuth-213 daughters of the actinium-225 with the chelator to reduce nephrotoxicity in the individual during the treatment thereby increasing the therapeutic index of the actinium-225 to improve the treatment for cancer. Further to this embodiment there is provided a method of administering a pharmacologically effective dose of a diuretic; and inhibiting renal uptake of francium-211 daughters of the actinium-225 with the diuretic to reduce nephrotoxicity in the individual during the treatment thereby increasing the therapeutic index of the actinium-225 to improve the treatment for the cancer.

In a related embodiment there is provided a method of improving radioimmunotherapeutic treatment of cancer in an individual, comprising administering a pharmacologically effective dose of a diuretic; administering an actinium-225 radioimmunoconjugate; and inhibiting renal uptake of francium-211 daughters of the actinium-225 with the diuretic to reduce nephrotoxicity in the individual during the treatment thereby increasing the therapeutic index of the actinium-225 to improve the treatment for the cancer.

For all these embodiments the chelators and the diuretics are described supra, as are the points of administration of the chelator and/or the diuretic during treatment. Again in these embodiments the ²²⁵Ac radioimmunoconjugate and the cancers treated are as described supra.

In yet another embodiment there is provided a method of increasing the therapeutic index of an actinium-225 radioimmunoconjugate during treatment of a pathophysiological condition in an individual comprising inhibiting renal uptake of at least one alpha particle-emitting daughter of actinium-225 whereby nephrotoxicity is reduced during the treatment thereby increasing the therapeutic index of the actinium-225 radioimmunoconjugate.

In an aspect of this embodiment the step of inhibiting renal uptake comprises administering a pharmacologically effective amount of an adjuvant comprising a chelator to scavenge the ²²⁵Ac daughters therewith or of a diuretic to inhibit reabsorption of the ²²⁵Ac daughters within a kidney, or a competitive metal blocker to prevent binding of said ²²⁵Ac daughters within a kidney or a combination thereof. An example of an ²²⁵Ac daughter scavenged by a chelator is bismuth-213. An example of an ²²⁵Ac daughter that is inhibited from reabsorbing into the kidneys is francium-211. An example of an ²²⁵Ac daughter that is prevented from binding within a kidney is ²¹³Bi.

In all embodiments and aspects thereof, the pathophysiological condition may be a cancer or an autoimmune disorder. The cancer may be a solid cancer, a disseminated cancer or a micrometastatic cancer. An example of a cancer is myeloid leukemia. Furthermore, the chelators, the diuretics, the competitive metal binders, the points of administration thereof during treatment, the ²²⁵Ac radioimmunoconjugate and the cancers treated are as described supra.

As used herein “radioimmunotherapy” shall refer to targeted cancer therapy in which a radionuclide is directed to cancer cells by use of a specific antibody carrier.

As used herein, “alpha particle” shall refer to a type of high-energy, ionizing particle ejected by the nuclei of some unstable atoms that are relatively heavy particles, but have low penetration.

As used herein, “radionuclide” shall refer to any element that emits radiation from its nucleus.

As used herein, “²²⁵Ac nanogenerator”shall refer to a nano-scale, in-vivo generator of alpha particle emitting radionuclide daughters, produced by the attachment of a chelated Actinium-225 atom to a monoclonal antibody.

Provided herein are methods of controlling renal uptake of actinium-225 daughters generated by an ²²⁵Ac nanogenerator during targeted radioimmunotherapy which accelerate the clearance of the alpha particle-emitting daughters from the body. Methods utilizing metal chelation, diuresis, or competitive metal blockade may be used as adjunct therapies to modify the potential nephrotoxicity of ²²⁵Ac daughters. Generally, a radioimmunoconjugate comprising an ²²⁵Ac nanogenerator will bind a targeted tumor cell. Upon binding the actinium-255 decays and delivers the alpha particle-emitting daughters to the cell to effect treatment. Once the decay cascade sequence begins, however, the daughter radiometals are no longer bound to the antibody and all daughters are not delivered to the targeted tumor cell. Thus, the daughters are free to accumulate in healthy tissues such as the kidneys causing toxicity.

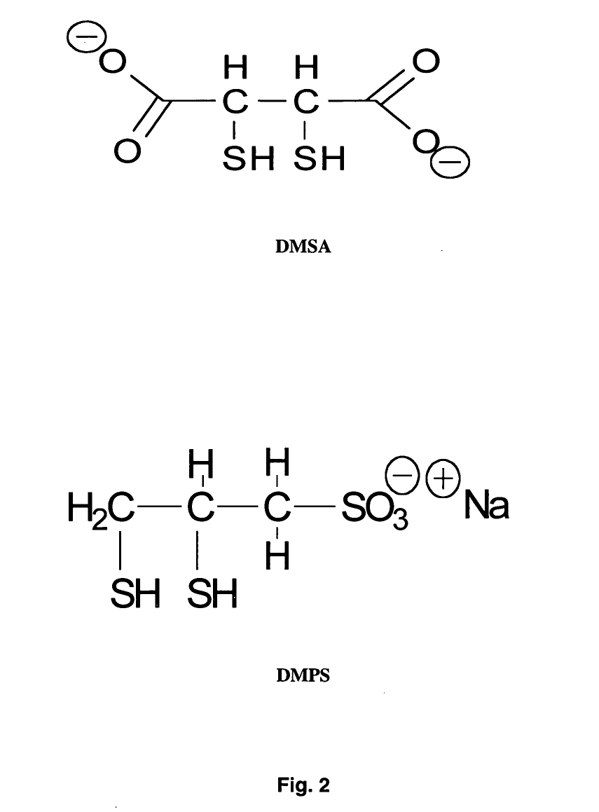

Chelated metals are protected and are, therefore, safe if detached from the antibody due to their rapid renal clearance. Chelators such as, but not limited to, the dithiol chelators 2,3 dimercapto-1-propane sulfonic acid (DMPS) and meso 2,3-dimercapto succinic acid (DMSA) shown in FIG. 2 or other chelators, e.g., ethylenediamine tetra-acetic acid (EDTA), diethylenetriamine pentaacetic acid (DTPA), calcium diethylenetriamine pentaacetic acid (Ca-DTPA), or zinc diethylenetriamine pentaacetic acid (Zn-DTPA),may be used to prevent the accumulation of free bismuth-213 daughters in the patient. Preferably, DMPS is used to chelate bismuth-213 daughters.

The present invention also provides methods of using diuretics to reduce renal uptake of francium-211 daughters and, by extension as a decay product thereof, bismuth-213 daughters into the nephron via inhibition of reabsorption of francium-211 through diuresis. Examples of such diuretics are furosemide, chlorthiazide, hydrochlorothiazide, bumex, or other loop diuretic. Additionally, competitive metal blockers may be used to compete with bismuth-213 for binding sites in the renal tubular cells of the kidney. Examples of a nonradioactive bismuth competitor are bismuth subnitrate or bismuth subcitrate.

Thus, as described herein, adjuvants, e.g., chelators, diuretics or competitive metal blockers, either individually or in combination, may be used as an adjunct chelating therapy to modify the nephrotoxicity of bismuth-213 and/or francium-211. Combination of adjuvant therapies results in cumulative effects over individual therapies. Therefore, nephrotoxicity is reduced during treatment and larger and more effective doses of the ²²⁵Ac nanogenerator may be administered. This may allow up to a doubling or more of the therapeutic index of such radiochemotherapeutics. As such, radioimmunotherapeutic treatment of pathophysiological conditions, such as but not limited to, cancers, e.g., leukemias, and autoimmune disorders are improved.

In the ²²⁵Ac nanogenerator the actinium-225 may be stably bound to a monoclonal antibody via a bifunctional chelant, such as a modified 1,4,7,10-tetraazacyclododecane-1,4,7,10-tetraacetic acid (DOTA) which chelates the actinium-225 while binding it to the monoclonal antibody. Although not limited to such, an example of a radioimmunoconjugate (RIC) suitable for targeted therapy of myeloid leukemia cells is the ²²⁵Ac nanogenerator [²²⁵Ac] DOTA-HuM195.

Additionally, the methods provided herein are more efficacious in reducing nephrotoxicity in patients with a higher tumor burden. The presence of high levels of a specific target tumor burden caused a decrease in the amount of circulating, untargeted antibody and, therefore, the systemically released daughters. Furthermore, the ²²⁵Ac nanogenerator comprises a monoclonal antibody that is internalized within the target tumor cells. Therefore, a sub-saturating amount of antibody, e.g., about 2-3 mg of HuM195, administered to a patient results in more of the generated daughters being retained inside the cancer cell because, theoretically,almost all of the antibody should be able to bind to the target cells and be internalized.

It is contemplated that the adjunct methods described herein may be used with targeted ²²⁵Ac nanogenerator radioimmunotherapy of pathophysiological conditions benefiting from ²²⁵Ac radioimmunotherapy. For example, the methods presented herein may be used in conjunction with radioimmunotherapeutic methods for treatment of solid cancers, disseminated cancers and micrometastatic cancers. Thus, leukemias, such as myeloid leukemia, may benefit from this adjunct therapy. It is further contemplated that other diseases or disorders for which ²²⁵Ac nanogenerator would be administered may benefit from these adjuvants. An example of such a disorder is an autoimmune disorder.

The adjuvants of the present invention may be administered prior to the ²²⁵Ac nanogenerator with continued administration after the radioimmunotherapeutic treatment. Routes of administration may be either oral or via injection, such as intravenous injection, and are well known to those of ordinary skill in the art.

It is also contemplated that administration of the adjuvant chelators, diuretics and competitive metal blockers is via an appropriate pharmaceutical composition. In such case, the pharmaceutical composition comprises the adjuvant and a pharmaceutically acceptable carrier. Such carriers are preferably non-toxic and non-therapeutic Preparation of such pharmaceutical compositions suitable for the mode of administration is well known in the art.

The adjuvants are administered in an amount to demonstrate a pharmacological effect, e.g., an amount to reduce nephrotoxicity due to bismuth-213 or francium-211 accumulation within the kidneys. An appropriate dosage may be a single administered dose or multiple administered doses. The doses administered optimize effectiveness against negative effects of radioimmunotherapeutic treatment. As with all pharmaceuticals, including the ²²⁵Ac nanogenerator described herein, the amount of the adjuvant administered is dependent on factors such as the patient, the patient's history, the nature of the cancer treated, i.e., solid or disseminated, the amount and specific activity of the actinium generator construct administered and the duration of the radioimmunotherapeutic treatment.

As the adjuvants of the present invention are approved and available for human use, the amounts administered would typically fall within recommended usage guidelines designated by the package inserts or by the general practice of medicine. For example, doses of DMPS may be in the recommended range of 0.1-1 mmol/kg/d for the treatment of heavy metal poisoning (64). An example of a dosing regimen for DMSA may be about 10 mg/kg every 8 hours and for DMPS may be 200-1500 mg/day in divided doses.

It is contemplated that use of the adjuvant therapies described herein would allow significant escalation of patient doses of actinium-225. A therapeutic dose of an adjuvant where the ratio of available adjuvant molecules to ²¹³Bi atoms or ²¹¹Fr atoms is substantially high provides for a significant reduction in nephrotoxicity. Therefore, with a capability to clear free actinium-225 daughters greater than the daughters generated for a given dose, higher doses of the ²²⁵Ac nanogenerator may be administered with a reduced risk of subsequent nephrotoxicity during treatment. A dose of about 0.5 μCi/kg to about 5.0 μCi/kg of actinium-225 may be used to treat the patient. A representative example is about 1 μCi/kg of actinium-225. However, determination of dosage of the adjuvants described herein and of the ²²⁵Ac nanogenerator is well within the skill of an artisan in the field and may be determined to be any therapeutically effective amount using at least the criteria discussed supra.

As described herein, the invention provides a number of therapeutic advantages and uses. The embodiments and variations described in detail herein are to be interpreted by the appended claims and equivalents thereof. The following examples are given for the purpose of illustrating various embodiments of the invention and are not meant to limit the present invention in any fashion.

### EXAMPLE 1

Animals

Female BALB/cand severe combined immunodeficient (SCID) mice, 4-12 weeks of age, were obtained from Taconic, Germantown, N.Y. Cynomologus monkeys were obtained. All animal studies were conducted according to the NIH Guide for the care and use of laboratory animals and were approved by the Institutional Animal Care and Use committee at Memorial Sloan Kettering Cancer Center.

### EXAMPLE 2

Preparation and Quality Control of Actinium-225 Labeled Antibodies

²²⁵Ac was conjugated to SJ25C1, a mouse anti-human CD19 IgG1 monoclonal antibody (Monoclonal Antibody Core Facility, Memorial Sloan Kettering Cancer Center) or HuM195, a humanized anti-CD33 IgG1 monoclonal antibody; (Protein Design Labs, Fremont, Calif.) using a two-step labelling method, as described previously (76). Routine quality control of the labeled antibody was performed using instant thin layer chromatography (ITLC) to estimate the radio-purity (62,77).

### EXAMPLE 3

Administration of Actinium-225 Nanogenerator to Mice

The mice were anesthetized and then injected intravenously in the retro-orbital venous plexus with 0.5 μCi of either ²²⁵Ac labeled HuM195 for chelation, diuresis and competitive metal blockade experiments or of ²²⁵Ac labeled SJ25C1 for tumor burden experiments. The injected volume was 100 μl. In order to detect adequate numbers of disintegrations in tissues by use of the gamma-counter, the injected doses of ²²⁵Ac nanogenerator, i.e., ˜30 μCi/kg, are much higher than the doses for human clinical trials with these adjuvants.

### EXAMPLE 4

Statistical Analysis

Graphs were constructed using Prism (Graphpad Software Inc., SanDiego, Calif.). Statistical comparisons between experimental groups were performed by either the Student's t-test (two-group comparison) or one-way ANOVA with Bonferroni's multiple comparison post-hoc test (three-group comparison). The level of statistical significance was set at p<0.05.

The inter-experiment variance in the tissue daughter activities at a given time-point was expected due to possible age-related variability in the capacity of the reticuloendothelial system to metabolize the labeled antibody. However, the intra-experiment variability within an experimental group was very small.

### EXAMPLE 5

Free Metal Scavenging with DMPS or DMSA

Animals received either 2,3-dimercapto-1-propanesulfonic acid (DMPS; Sigma, St. Louis, Mo.) or meso-2,3-dimercaptosuccinic acid (DMSA; Sigma, St. Louis, Mo.) in drinking water (1.2 mg/ml and 1.5 mg/ml, respectively), starting one day before injection with ²²⁵Ac nanogenerator and continued until the animals were sacrificed. The control animals received regular drinking water. Animals (n=5 per group) were sacrificed at 6 and 72 hours post-injection by carbon-dioxide asphyxiation.

Samples of blood taken by cardiac puncture, of kidneys, of liver and of small intestine were removed. The organs were washed in distilled water, blotted dry on gauze, weighed, and the activity of ²²¹Fr (185-250 keV window) and ²¹³Bi (360-480 keV window) was measured using a gamma counter (COBRA II, Packard Instrument Company, Meriden, Conn.). Samples of the injectate (100 μl) were used as decay correction standards. Adjustment was made for the small percentage of bismuth activity that counted in the francium activity window. Percentage injected dose of ²²⁵Ac, ²²¹Fr and ²¹³Bi per gram of tissue weight (% ID/g) was calculated for each animal at the time of sacrifice, using the equation (78):

A₂₍₀₎=[A₂−A_{2(eq)}·(e^{-λ2t}−e^{-λ1t})]·e^{λ2t}

where λ1 and λ2 are the decay constants of Ac and Bi, respectively. The mean % ID/g was determined for each experimental group.

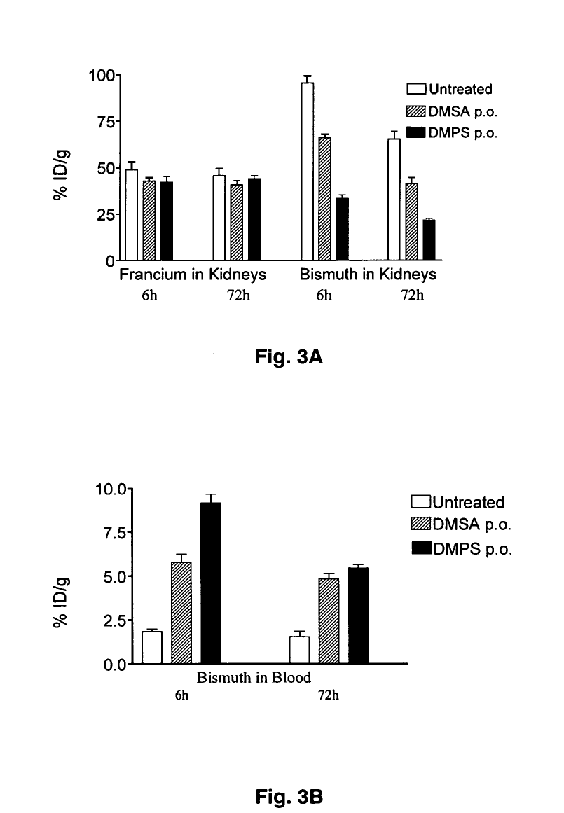

The renal ²¹³Bi activity differed significantly between the DMPS or DMSA treated groups and untreated controls at 6 hours (ANOVA, p<0.0001) and 72 hours (ANOVA, p<0.0001) post-injection with the ²²⁵Ac nanogenerator (FIG. 3A). The 6 hour renal ²¹³Bi activity in the control group was 95.7±3.8% ID/g, which was reduced to 38.6±5.5% ID/g and 66.0±1.9% ID/g in DMPS and DMSA treated groups, respectively. A similar reduction in the renal 213Bi activity was observed at 72 hours post-injection of 66.7±7.9% ID/g in controls versus 21.7±2.1% ID/g and 41.4±7.3 in DMPS and DMSA treated groups, respectively. DMPS was significantly more effective than DMSA in preventing the renal ²¹³Bi accumulation at both time-points (6 h, p<0.001;72 h, p<0.001). The renal ²²¹Fr activity, however, was not significantly different between the experimental groups at either 6 hours (ANOVA, p=0.39) or 72 hours (ANOVA, p=0.20) post-injection (FIG. 3A).

As shown in FIG. 3B, the mean blood ²¹³Bi activity was higher (6 h, ANOVA p<0.0001;72 h, ANOVA p<0.0001) in the DMPS (9.2±0.5% ID/g and 5.5±0.1% ID/g at 6 and 72 hours, respectively) and DMSA (5.8±0.5% ID/g and 4.8±0.6% ID/g at 6 and 72 hours, respectively) treated groups as compared to the controls with 1.8±0.1% ID/g and 1.5±0.7% ID/g at 6 and 72 hours, respectively. However, the blood ²²¹Fr activity was unaltered by chelation therapy (data not shown). Similar results were seen with calcium-diethylenetriamine pentaacetate (Ca-DTPA), but it was less effective than DMPS in reducing the renal ²¹³Bi activity (data not shown).

In plasma the dithiol chelators are transported free or as disulfides with plasma proteins and non-protein sulfhydryl compounds, e.g. cysteine (79). In human plasma, DMPS has been shown to form non-protein sulfhydryls to a greater extent at 37%, than DMSA at 8%. Therefore, DMPS is thought to be more reactive in plasma than DMSA (79). Also, it is believed that the presence of charged carboxyl groups impede the transport of DMSA through cell membranes (80).

These factors may account for the greater effectiveness of DMPS in reducing the renal ²¹³Bi uptake, as compared to DMSA. DMPS, being more reactive, is rapidly oxidized in aqueous solutions to form di-sulfides (81). However, a loss of efficacy was not observed when DMPS was administered in drinking water. This possibly is due to disulfide reduction in the renal tubular cells by a glutathione-disulfide exchange reaction, to yield the parent drug. This effect has been shown in previous studies (79).

The increase in the blood ²¹³Bi activity with chelation therapy may have resulted from the chelation and retention of ²¹³Bi generated in blood from the circulating ²²⁵Ac nanogenerators or from the extraction of tissue ²¹³Bi into the blood stream. The circulating chelator-²¹³Bi complex is not expected to cause any significant toxicity due to the short path length of alpha particles (50). In contrast, the reduction in the renal ²¹³Bi activity is critical to the safety of the ²²⁵Ac nanogenerators.

### EXAMPLE 6

Diuretic Therapy

Mice were randomized to furosemide treatment, chlorthiazide (CTZ) treatment or no treatment(control)groups (5 animals per group). Furosemide and CTZ were administered intraperitoneally (i.p.). The loading doses of furosemide and CTZ were 250 mg/kg and 750 mg/kg respectively, administered one hour before ²²⁵Ac nanogeneratorinjection. The maintenance doses were 100 mg/kg and 300 mg/kg, respectively, administered 12 hours and 24 hours after the loading dose. The controls were injected with an equal volume of saline (vehicle).

Alternatively, mice received DMPS (1.2 mg/ml in drinking water) and either furosemide or CTZ i.p using the same dose schedule as above. The controls received regular drinking water and were injected with an equal volume of saline. The animals were sacrificed at 24 hours post-injection with the labeled antibody and the mean activity (% ID/g) of ²²⁵Ac, ²²¹Fr and ²¹³Bi in blood and kidneys was calculated for each experimental group, as described above.

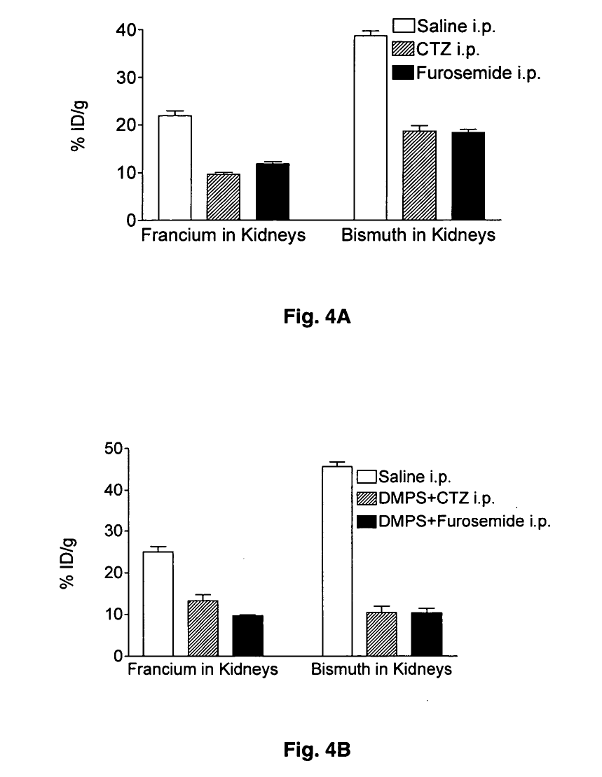

Diuretic therapy prevented the renal accumulation of both ²²¹Fr and ²¹³Bi (FIG. 4A). The 24 hour renal ²²¹Fr activity differed significantly (ANOVA, p<0.0001) between the experimental groups (21.9±1.0% ID/g in controls versus 11.8±0.4% ID/g and 9.7±0.4% ID/g in furosemide and CTZ treated groups, respectively). Similarly, the 24 hour renal ²¹³Bi activity was 38.7±1.0% ID/g in the controls versus 18.3±0.6% ID/g and 18.6±1.6% ID/g in furosemide and CTZ treated groups, respectively(ANOVA, p<0.0001). However, the renal ²²¹Fr and ²¹³Bi activities were not significantly different between the two treated groups (Bonferroni's post-hoc analysis, p>0.05 for both ²²¹Fr and ²¹³Bi activities).

Furthermore, the combination of DMPS with a diuretic, furosemide or CTZ, caused a greater reduction of ˜75-80% in the renal ²¹³Bi activity than seen with DMPS or diuretics alone (FIGS. 4A-4B). The 24 hour renal ²¹³Bi activity was 45.7±1.0% ID/g in controls versus 10.4±1.0% ID/g and 10.5±1.5% ID/g in DMPS+furosemide and DMPS+CTZ groups, respectively (ANOVA, p<0.0001). The reduction in the renal ²²¹Fr accumulation, however, was similar to that seen with diuretic treatment (25.7±1.3% ID/g in controls versus 9.7±0.4% ID/g and 13.3±1.4% ID/g in DMPS+furosemide and DMPS+CTZ groups, respectively (ANOVA, p<0.0001).

Different classes of diuretics inhibit the tubular reabsorption of the alkali metals, Na⁺ or K⁺ or both, although they differ in their potency, mechanism and site of action within the nephron. Furosemide and CTZ act, respectively, in the ascending limb of Henle's loop and distal convoluted tubule of the nephron (82). The significant drop in the renal ²²¹Fr activity with furosemide and CTZ possibly is due to an inhibition of the renal tubular reabsorption of ²²¹Fr which is an alkali metal and is, therefore, expected to behave like Na⁺ and K⁺. Since ²¹³Bi is generated from ²²¹Fr, a decrease in the renal ²¹³Bi ensued. Furthermore, the combination of DMPS with a diuretic, e.g., furosemide or CTZ, resulted in an even greater reduction in renal ²¹³Bi activity than seen with DMPS or the diuretics alone. The administered doses of furosemide and CTZ were scaled from previously published literature on their ED₅₀ in mice. The doses exceed the human therapeutic doses as there is a species difference in the ED₅₀ of these drugs (83).

### EXAMPLE 7

Competitive Metal Blockade

Mice (5 per group) were injected i.p. with 200 μl of 1% bismuth subnitrate (BSN; Sigma, St. Louis, Mo.) suspension (100 mg/kg) or an equal volume of saline (controls) 4 hours before ²²⁵Ac nanogenerator injection. These animals were sacrificed at 6 hours post-injection with the ²²⁵Ac nanogenerator. Alternatively, mice were injected i.p. with 200 μl of 1% BSN suspension, 4 hours before and 8 and 20 hours after ²²⁵Ac nanogenerator injection (n=5) or an equal volume of saline (n=5). These animals were sacrificed 24 hours after ²²⁵Ac nanogenerator injection. The mean % ID/g of ²²⁵Ac, ²²¹Fr and ²¹³Bi in blood and kidneys at sacrifice-time was calculated for each experimental group.

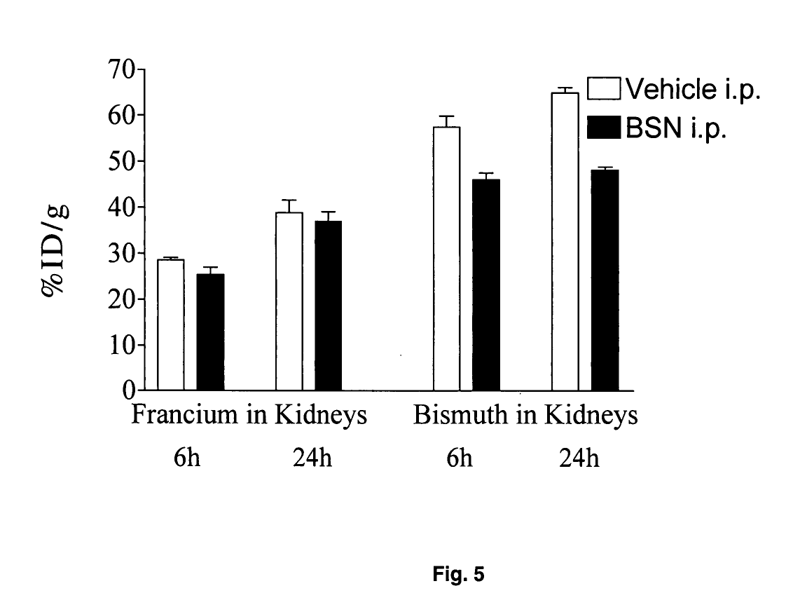

Competitive blockade of ²¹³Bi binding-sites in the renal tubular cells by non-radioactive bismuth resulted in a moderate, but significant, reduction in the renal ²¹³Bi activity at both 6 hour (p=0.004) and 24 hour (p<0.0001) time-points (FIG. 5). Renal ²¹³Bi activity at 6 and 24 hours post-injection was 57.5±2.4% ID/g and 64.9±1.2% ID/g, respectively in controls versus 46.1±1.4% ID/g and 48.2±0.6% ID/g, respectively in BSN treated animals. As expected, the renal ²²¹Fr activity was unaltered (FIG. 5) at either time-point (6 hours, p=0.10;24 hours, p=0.61).

### EXAMPLE 8

Effect of DMPS on Tumor Burden

Disseminated human Daudi lymphoma (84) treated with ²²⁵Ac labeled anti-CD19, was used as the model system. SCID mice, 10-12 weeks old, were randomized to “low tumor burden” or 7 days growth of tumor, “high tumor burden” or 30 days growth of tumor or “high tumor burden+DMPS” group or 30 days growth of tumor and treated with 1.2 mg/ml DMPS in drinking water, starting one day before injection with ²²⁵Ac nanogenerator. All mice were injected intravenously with 5×10⁶ Daudi lymphoma cells in 0.1 ml phosphate buffered saline (PBS). The “low burden” animals were injected with the tumor cells 23 days after the “high burden” ones. The animals were checked daily for the onset of hind-leg paralysis. 30 days after injection of tumor cells in the “high burden” animals and 7 days after injection for the “low burden” group, all animals were injected retro-orbitally with 0.5 μCi of ²²⁵Ac labeled SJ25C1 in 100 μl.

The animals (5 per group) were sacrificed at 24 hours post-injection and the mean ²²⁵Ac, ²²¹Fr and ²¹³Bi activity (% ID/g) in blood, femurs and kidneys was calculated for each experimental group. The % of human-CD20 positive cells in the femoral bone marrow was estimated in one representative animal from the “high and low burden” groups by flow cytometric staining with phycoerythrin (PE)-conjugated anti-human CD20 (BD, San Jose, Calif.) and compared to that of a non tumor-bearing mouse of the same strain.

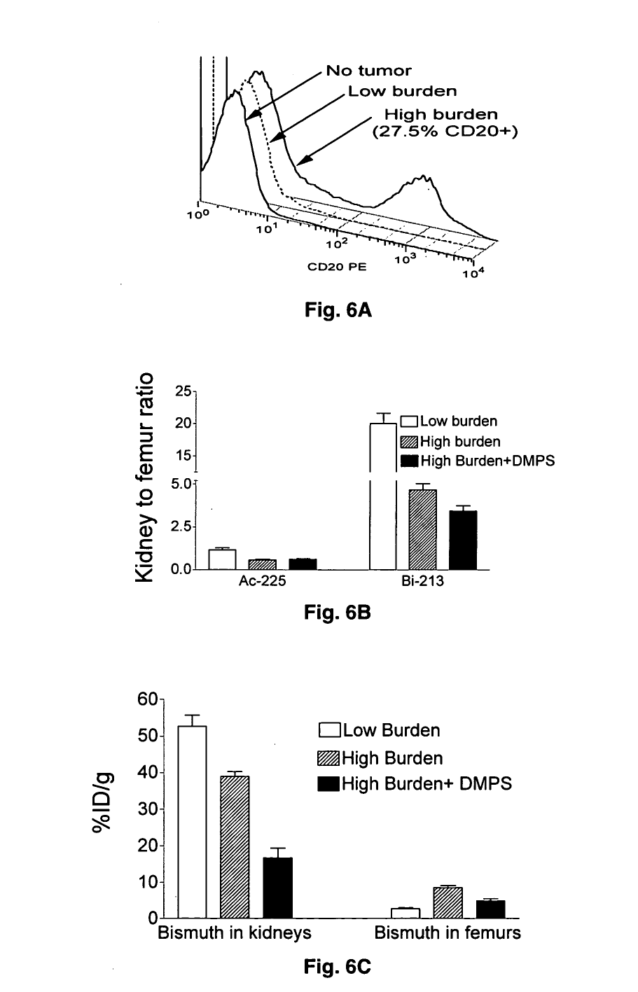

The expression of CD19 and CD20 antigens and binding of the antibody (SJ25C1) to CD19 on Daudi cells were confirmed by flow cytometry before injecting the tumor in animals. The percentage of target lymphoma cells, i.e., bone marrow cells positive for human CD20, in one representative “low burden” and “high burden” animal were 0.12% and 27.5%, respectively (FIG. 6A). Due to higher localization of the labeled antibody (²²⁵Ac activity) to the femurs, the kidneys to femur activity ratios for ²²⁵Ac were significantly lower (p<0.0001) in the groups with higher tumor burden (FIG. 6B).

As demonstrated in FIG. 6C, the presence of a higher tumor burden resulted in a significant decrease in the renal ²¹³Bi activity, (52.6±3.1% ID/g, in “low burden” versus 38.8±1.3% ID/g in “high burden” animals; p=0.003), which was reduced further by DMPS treatment (16.7±2.7% ID/g; p<0.0001 compared to untreated “high burden” group and p<0.0001 compared to “low burden” group). The femur ²¹³Bi activity was significantly higher (p<0.0001) in the untreated “high burden” group (8.5±0.5% ID/g) as compared to the “low burden” group (2.7±0.3% ID/g). However, DMPS treated “high burden” animals had lower ²¹³Bi activity (p=0.002) in the femurs (4.8±0.6% ID/g) than untreated “high burden” animals (FIG. 6C). The ratio of kidney to femur activity for ²¹³Bi was significantly lower (p<0.0001) in the high tumor burden group (FIG. 6B).

The presence of high levels of a specific target, i.e., tumor burden, caused a decrease in the amount of circulating, untargeted antibody and, therefore, the systemically released daughters. This translated to an increase in the activity of ²²⁵Ac and its radioactive daughters in the femurs where the tumor resided and a corresponding decrease in their activities in the kidneys. The effect may have been blunted by the large dose of antibody used and the low specific activity of the radioimmunoconjugate as, approximately, 1 out of 1000 antibodies were labeled with ²²⁵Ac.

Based on the number of available CD19 sites per Daudi cell, 120 million tumor cells, which is an estimated tumor load in a “high burden animal”, are expected to maximally absorb approximately 1.2 μg of the antibody, whereas 6.7 μg of the antibody was injected per animal. This translates to an excess of injected antibodies as compared to the available binding sites. A typical acute myeloid leukemia patient has approximately 10¹²leukemia cells and based on the available CD33 sites, approximately 5 mg of HuM195 could be absorbed. However, administering sub-saturating amounts, i.e., about 2-3 mg of antibody per patient would yield a more pronounced reduction in the renal daughter accumulation is expected.

DMPS treatment further reduced the renal ²¹³Bi accumulation in animals that bore the target tumor. Additionally, a reduction in the femur ²¹³Bi activity was seen in these animals. However, despite the reduction in the ²¹³Bi activity in the femurs, the kidney to femur activity ratio in these animals for ²¹³Bi was, in fact, significantly lower. This is because of a greater relative reduction in the ²¹³Bi accumulation in kidneys than in the femurs. Free bismuth has been shown to accumulate in the femurs even in the absence of a bone marrow tumor (64). Therefore, the ²¹³Bi activity in the femurs cannot be entirely accounted for by the ²¹³Bi inside the tumor cells. The reduction in the femur ²¹³Bi activity may be due to its scavenging from the tumor cells or the femurs. It also could be due to scavenging of free ²¹³Bi produced on the surface of the tumor cells as a result of the attachment of the labeled antibody.

### EXAMPLE 9

In vivo Biodistribution of [Ac]Hum195 at 24 Hours

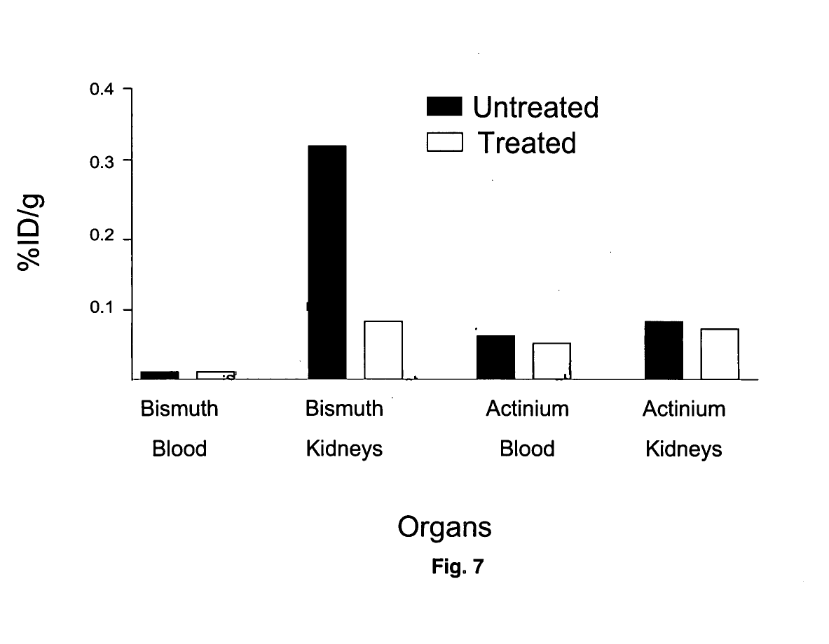

Two cynomolgus monkeys weighing about 7 kg were injected with 25 μCi of Ac-225 nanogenerators on HuM195 antibodies. One monkey received water and the other received DMPS in water for 24 hours and one dose of DMPS intravenously 90 min before sacrifice. At 24 hours the two monkeys were sacrificed and the kidneys examined for Bi-213 daughters. A 70% reduction in Bi-213 in the kidneys of the treated monkey was found (FIG. 7).

The following references are cited herein:

1. Scheinberg D A, Maslak P M, Weiss M. Acute Leukemia. In: Cancer: Principles and Practice of Oncology; pp 2404-2432; DeVita V., et al. Eds.; Lipincott-Raven, Publishers, New York (2001).

2. Scheinberg et al., Cancer Res. 42:44-49 (1982).

3. Scheinberg et al., Cancer Res. 43:265-272 (1983).

4. Scheinberg et al., J Clin Oncol 9:478-490 (1991).

5. Nadler et al., Cancer Res 40:3147-54 (1980).

6. Shawler et al., Cancer Res 44:5921-5927 (1984).

7. Ritz et al., Blood 58:141-152 (1981).

8. Scheinberg et al., Science 215:1511-1513 (1982).

9. Gansow et al., Generator produced Bi-212 chelated to chemically modified monoclonal antibodies for use in radiotherapy. In: Radionuclide Generators: New Systems for Nuclear Medicine Application. F F Knapp, T A Butler, Eds. ACS. Washington, D.C. (1984).

10. Kassis et al., Radiat Res 84:407-425 (1980).

11. Sastry K S R, Rao D V. Dosimetry of low energy electrons. Rao D V, Chandra R, Graham M C. eds. In: Physics of Nuclear Medicine, American Association of Physicists in Medicine (1984).

12. Houghton et al., Sem Oncol 13:165-179 (1986).

13. Miller et al., Lancet 2:226-230 (1981).

14. Foon et al., Blood 64:1085-1093 (1984).

15. Dillman et al., J Clin Oncol 2:881-891 (1984).

16. Scheinberg et al., J Clin Oncol 8:792-803 (1990).

17. Denardo et al., J Clin Oncol 16:3246-3256 (1998).

18. Hale et al., Lancet 2:1394-1399 (1988).

19. Schwartz et al., J Clinical Oncol 11:294-303 (1993).

20. Matthews et al., Blood 85:1122-1131 (1995).

21. Jurcic et al., Leukemia 9:244-248 (1995).

22. Czuczman et al., J Clin Oncol 11:2021-2029 (1993).

23: Kaminski et al., J Clin Oncl 14:1974-1981 (1996).

24. McLaughlin et al., J Clin Oncol 92:2825-2833 (1998).

25. Czuczman et al., J Clin Oncol 17:268-276 (1999).

26. Knox et al., Clin Cancer Res 2:457-470 (1996).

27. Keating et al., Blood 94 (suppl):705 (1999).

28. Sievers et al., Blood 93:3678-2684 (1999).

29. Caron et al., Blood 83:1760-1768 (1994).

30. Jurcic et al., Blood 98(9):2651-2656 (2001).

31. Feldman et al., Proceedings of ASCO (2002).

32. Bernstein et al., J Clin Invest 79:1153 (1987).

33. Tanimoto et al., Leukemia 3:339-348 (1989).

34. Scheinberg et al., Leukemia 3:440-445 (1989).

35. Griffin et al., Leuk Res 8:521-534 (1984).

36. Applebaum et al.,. Transplantation 54:829-833 (1992).

37. Bloomer et al., Science 212:340-341 (1981).

38. Garg et al., Cancer Res 50:3514-3520 (1990).

39. Howell et al., Radiation Protection Dosimetry 31:325-328.

40. Mackliss et al., Radiation Research 130:220-226 (1992).

41. Humm et al.,. Radiation Research 134:143-150 (1993).

42. Geerlings et al., Nuclear Medicine Comm. 14:121-125 (1993).

43. McDevitt et al., Eur. J. Nuc. Med., 25 (9), 1341-1351 (1998).

44. Vaidyanathan G, Zalutsky M R. Targeted therapy using alpha emitters. Phys Med Biol 1:1905-1914 (1994).

45. Behr, et al., Cancer Res. 59, 2635-43 (1999).

46. Wilber D A. Antibody, Immunoconjugates and Radiopharmaceuticals 4:85-97 (1991).

47. Kaspersen et al., Nuclear Medicine Communications 16:468-476 (1995).

48. Brechbiel et al., J. Chem. Soc. Perkin Trans. 1, 1173-1178 (1992).

49. McDevitt et al., Cancer Res. 60:6095-6102 (2000b).

50. Jurcic et al., Blood 100(4):1233-9 (2002).

51. Sgouros, et al., J. of Nucl. Med. 40 (11), 1935-1946 (1999).

52. McDevitt et al., Cell Death Differ 9(6):593-4 (2002).

53. Chang et al., Mol Cancer Ther 1(7):553-63 (2002).

54. Kozak et al., Proc Natl Acad Sci U S A 83(2):474-8 (1986).

55. Bethge et al., Blood 101(12):5068-75 (2003).

56. Yao et al., J Nucl Med 42(10):1538-44 (2001).

57. Waldmann T A. Immunotherapy: past, present and future. Nat Med 9(3):269-77 (2003).

58. Mulford et al., Expert Opin Biol Ther 4(1):95-105 (2004).

59. Zalutsky et al., Curr Pharm Des 6(14):1433-55 (2000).

60. Raju et al., Radiat Res 128(2):204-9 (1991).

61. McDevitt et al., Science 294(5546):1537-40 (2001).

62. Borchardt et al., Cancer Res 63(16):5084-90 (2003).

63. Meiderer et al., J Nucl Med 2004

64. Jones et al., Nucl Med Biol 23(2):105-13 (1996).

65. Sgouros et al., J Nucl Med 34(3):422-30 (1993).

66. Szymanska et al., Biochem Pharmacol 26(3):257-8 (1997).

67. Russ et al., Radiat Res 63(3):443-54 (1975).

68. Slikkerveer et al., Med Toxicol Adverse Drug Exp 4(5):303-23 (1989).

69. Slikkerveer et al., J Lab Clin Med 119(5):529-37 (1992).

70. Yung et al., Pharmacol Biochem Behav 21 Suppl 1:71-5 (1984).

71. Basinger et al., J Toxicol Clin Toxicol 20(2):159-65 (1983).

72. Slikkerveer et al., Analyst 123(1):91-2 (1998).

73. Bruenger et al., Int J Radiat Biol 60(5):803-818 (1991).

74. Breitenstein et al., The U.S. Experience 1958-1987,″ in: The Medical Basis of Radiation Accident Preparedness. 2 ed: Elsevier Science Publishing Co., Inc., 397-406 (1990).

75. Reyes et al., Cardiovasc Drugs Ther 13(5):371-98 (1999).

76. McDevitt et al., Appl Radiat Isot 57(6):841-7 (2002).

77. Nikula et al., J Nucl Med 40(1):166-76 (1999).

78. Mirzadeh et al., Radiochimica Acta 60:1-10 (1993).

79. Maiorino et al., J Pharmacol Exp Ther 277(1):375-84 (1996).

80. Aposhian et al., Annu Rev Pharmacol Toxicol 30:279-306 (1990).

81. Aposhian et al., Life Sci 31(19):2149-56 (1982).

82. Puschett J B. Cardiology 84 Suppl 2:4-13 (1994).

83. Hesdorffer et al., Ann Neurol 2001;50(4):458-62 (2001).

84. Ghetie et al., Blood 83(5):1329-36 (1994).

Any patents or publications mentioned in this specification are indicative of the levels of those skilled in the art to which the invention pertains. Further, these patents and publications are incorporated by reference herein to the same extent as if each individual publication was specifically and individually incorporated by reference.

One skilled in the art will appreciate readily that the present invention is well adapted to carry out the objects and obtain the ends and advantages mentioned, as well as those objects, ends and advantages inherent herein. The present examples, along with the methods, procedures, treatments, molecules, and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of reducing nephrotoxicity in an individual during radioimmunotherapeutic treatment of a pathophysiological condition, comprising:
administering a pharmacologically effective dose of at least one adjuvant effective for preventing accumulation of a metal in kidneys;
administering an actinium-225 radioimmunoconjugate to treat the pathophysiological condition; and
preventing accumulation of alpha particle-emitting daughters of said actinium-225 within the kidneys of the individual via interaction between said adjuvant and said ²²⁵Ac daughters or the kidney tissue or a combination thereof thereby reducing nephrotoxicity during the radioimmunotherapeutic treatment.

2. The method of claim 1, wherein said adjuvant(s) is administered prior to administering said actinium-225 radioimmunoconjugate, said adjuvant(s) continuing to be administered after said actinium-225 radioimmunoconjugate.

3. The method of claim 1, wherein said adjuvant is a chelator, a diuretic, a competitive metal blocker, or a combination thereof.

4. The method of claim 3, wherein said chelator is 2,3 dimercapto-1-propane sulfonic acid, meso 2,3-dimercapto succinic acid, diethylenetriamine pentaacetic acid, calcium diethylenetriamine pentaacetic acid, or zinc diethylenetriamine pentaacetic acid.

5. The method of claim 3, wherein said diuretic is furosemide, chlorthiazide, hydrochlorothiazide, bumex or other loop diuretic.

6. The method of claim 3, wherein said competitive metal blocker is bismuth subnitrate or bismuth subcitrate.

7. The method of claim 1, wherein said ²²⁵Ac daughter is bismuth-213, francium-221 or a combination thereof.

8. The method of claim 1, wherein said actinium-225 radioimmunoconjugate comprises an actinium-225 bifunctional chelant and a monoclonal antibody.

9. The method of claim 8, wherein said actinium-225 radioimmunoconjugate is [²²⁵Ac] DOTA-HuM195.

10. The method of claim 1, wherein said pathophysiological condition is a cancer or an autoimmune disorder.

11. The method of claim 1, wherein said cancer is a solid cancer, a disseminated cancer or a micrometastatic cancer.

12. The method of claim 11, wherein said cancer is myeloid leukemia.

13. A method of reducing nephrotoxicity in an individual during radioimmunotherapeutic treatment a pathophysiological condition, comprising:
administering a pharmacologically effective dose of a chelator;
administering an actinium-225 radioimmunoconjugate to treat the cancer; and
preventing accumulation of bismuth-213 daughters of said actinium-225 within the kidneys of the individual by scavenging thereof with said chelator thereby reducing nephrotoxicity during the radioimmunotherapeutic treatment.

14. The method of claim 13, wherein said chelator is administered prior to administering said ²²⁵Ac radioimmunoconjugate, said chelator continuing to be administered after said ²²⁵Ac radioimmunoconjugate.

15. The method of claim 13, wherein said chelator is 2,3 dimercapto-1-propane sulfonic acid, meso 2,3-dimercapto succinic acid, diethylenetriamine pentaacetic acid, calcium diethylenetriamine pentaacetic acid, or zinc diethylenetriamine pentaacetic acid.

16. The method of claim 13, further comprising:
administering a pharmacologically effective dose of a diuretic; and
preventing accumulation of francium-211 daughters of said actinium-225 within the kidneys of the individual by inhibiting reabsorption of francium-211 therein with said diuretic thereby reducing nephrotoxicity during the radioimmunotherapeutic treatment.

17. The method of claim 16, wherein said diuretic is administered prior to administering said ²²⁵Ac radioimmunoconjugate, said diuretic continuing to be administered after said ²²⁵Ac radioimmunoconjugate.

18. The method of claim 16, wherein said diuretic is furosemide, chlorthiazide, hydrochlorothiazide, bumex, or other loop diuretic.

19. The method of claim 13, wherein said ²²⁵Ac radioimmunoconjugate comprises an actinium-225 bifunctional chelant and a monoclonal antibody.

20. The method of claim 19, wherein said ²²⁵Ac radioimmunoconjugate is [²²⁵Ac] DOTA-HuM195.

21. The method of claim 13, wherein said pathophysiological condition is a cancer or an autoimmune disorder.

22. The method of claim 21, wherein said cancer is a solid cancer, a disseminated cancer or a micrometastatic cancer.

23. The method of claim 22, wherein said cancer is myeloid leukemia.

24. A method of reducing nephrotoxicity in an individual during radioimmunotherapeutic treatment of a pathophysiological condition, comprising:
administering a pharmacologically effective dose of a diuretic;
administering an actinium-225 radioimmunoconjugate to treat the cancer; and
preventing accumulation of francium-211 daughters of said actinium-225 within the kidneys of the individual by inhibiting reabsorption of francium-211 therein with said diuretic thereby reducing nephrotoxicity during the radioimmunotherapeutic treatment.

25. The method of claim 24, wherein said diuretic is administered prior to administering said ²²⁵Ac radioimmunoconjugate, said diuretic continuing to be administered after said ²²⁵Ac radioimmunoconjugate.

26. The method of claim 24, wherein said diuretic is furosemide, chlorthiazide, hydrochlorothiazide, bumex, or other loop diuretic.

27. The method of claim 24, wherein said ²²⁵Ac radioimmunoconjugate comprises an actinium-225 bifunctional chelant and a monoclonal antibody.

28. The method of claim 27, wherein said ²²⁵Ac radioimmunoconjugate is [²²⁵Ac] DOTA-HuM195.

29. The method of claim 24, wherein said pathophysiological condition is a cancer or an autoimmune disorder.

30. The method of claim 29, wherein said cancer is a solid cancer, a disseminated cancer or a micrometastatic cancer.

31. The method of claim 30, wherein said cancer is myeloid leukemia.

32. A method of improving radioimmunotherapeutic treatment of cancer in an individual, comprising:
administering a pharmacologically effective dose of a chelator;
administering an actinium-225 radioimmunoconjugate; and
scavenging bismuth-213 daughters of the actinium-225 with said chelator to reduce nephrotoxicity in the individual during the treatment thereby increasing the therapeutic index of the actinium-225 to improve the treatment for said cancer.

33. The method of claim 32, wherein said chelator is administered prior to administering said ²²⁵Ac radioimmunoconjugate, said chelator continuing to be administered after said ²²⁵Ac radioimmunoconjugate.

34. The method of claim 32, wherein said chelator is 2,3 dimercapto-1-propane sulfonic acid, meso 2,3-dimercapto succinic acid, diethylenetriamine pentaacetic acid, calcium diethylenetriamine pentaacetic acid, or zinc diethylenetriamine pentaacetic acid.

35. The method of claim 32, further comprising:
administering a pharmacologically effective dose of a diuretic; and
inhibiting renal uptake of francium-211 daughters of the actinium-225 with said diuretic to reduce nephrotoxicity in the individual during the treatment thereby increasing the therapeutic index of the actinium-225 to improve the treatment for-said cancer.

36. The method of claim 35, wherein said diuretic is administered prior to administering said ²²⁵Ac radioimmunoconjugate, said diuretic continuing to be administered after said ²²⁵Ac radioimmunoconjugate.

37. The method of claim 35, wherein said diuretic is furosemide, chlorthiazide, hydrochlorothiazide, bumex, or other loop diuretic.

38. The method of claim 35, wherein said ²²⁵Ac radioimmunoconjugate comprises an actinium-225 bifunctional chelant and a monoclonal antibody.

39. The method of claim 38, wherein said ²²⁵Ac radioimmunoconjugate is [²²⁵Ac] DOTA-HuM195.

40. The method of claim 35, wherein said cancer is a solid cancer, a disseminated cancer or a micrometastatic cancer.

41. The method of claim 40, wherein said cancer is myeloid leukemia.

42. A method of improving radioimmunotherapeutic treatment of cancer in an individual, comprising:
administering a pharmacologically effective dose of a diuretic;
administering an actinium-225 radioimmunoconjugate; and
inhibiting renal uptake of francium-211 daughters of the actinium-225 with said diuretic to reduce nephrotoxicity in the individual during the treatment thereby increasing the therapeutic index of the actinium-225 to improve the treatment for said cancer.

43. The method of claim 42, wherein said diuretic is administered prior to administering said ²²⁵Ac radioimmunoconjugate, said diuretic continuing to be administered after said ²²⁵Ac radioimmunoconjugate.

44. The method of claim 42, wherein said diuretic is furosemide, chlorthiazide, hydrochlorothiazide, bumex, or other loop diuretic.

45. The method of claim 42, wherein said ²²⁵Ac radioimmunoconjugate comprises an actinium-225 bifunctional chelant and a monoclonal antibody.

46. The method of claim 45, wherein said ²²⁵Ac radioimmunoconjugate is [²²⁵Ac] DOTA-HuM195.

47. The method of claim 42, wherein said cancer is a solid cancer, a disseminated cancer or a micrometastatic cancer.

48. The method of claim 47, wherein said cancer is myeloid leukemia.

49. A method of increasing the therapeutic index of an actinium-225 radioimmunoconjugate during treatment of a pathophysiological condition in an individual comprising:
inhibiting renal uptake of at least one alpha particle-emitting daughter of actinium-225 whereby nephrotoxicity is reduced during the treatment thereby increasing the therapeutic index of said actinium-225 radioimmunoconjugate.

50. The method of claim 49, wherein inhibiting renal uptake of said ²²⁵Ac daughter(s) comprises:
administering a pharmacologically effective amount of an adjuvant comprising:
a chelator to scavenge said ²²⁵Ac daughters therewith; or
a diuretic to inhibit reabsorption of said ²²⁵Ac daughters within a kidney; or
a competitive metal blocker to prevent binding of said ²²⁵Ac daughters within a kidney; or
a combination thereof.

51. The method of claim 50, wherein said chelator and/or said diuretic and/or said competitive metal blocker are administered prior to treatment with said actinium-225 radioimmunoconjugate, said chelator and/or said diuretic continuing to be administered after said actinium-225 radioimmunoconjugate is administered to the individual.

52. The method of claim 50, wherein said chelator is 2,3 dimercapto-1-propane sulfonic acid, meso 2,3-dimercapto succinic acid, diethylenetriamine pentaacetic acid, calcium diethylenetriamine pentaacetic acid, or zinc diethylenetriamine pentaacetic acid.

53. The method of claim 50, wherein said diuretic is furosemide, chlorthiazide, hydrochlorothiazide, bumex, or other loop diuretic.

54. The method of claim 50, wherein said competitive metal blocker is bismuth subnitrate or bismuth subcitrate.

55. The method of claim 50, wherein said chelator scavenges the ²²⁵Ac daughter bismuth-213.

56. The method of claim 50, wherein said diuretic inhibits reabsorption of the ²²⁵Ac daughter francium-211.

57. The method of claim 50, wherein said competitive metal binder prevents binding of the ²²⁵Ac daughter bismuth-213.

58. The method of claim 49, wherein said actinium-225 radioimmunoconjugate is [²²⁵Ac] DOTA-HuM195.

59. The method of claim 49, wherein said pathophysiological condition is a cancer or an autoimmune disorder.

60. The method of claim 59, wherein said cancer is a solid cancer, a disseminated cancer or a micrometastatic cancer.

61. The method of claim 60, wherein said cancer is myeloid leukemia.
